(19) United States
(12) Patent Application Publication
(10) Pub. No.: US 2017/0173245 A1
(43) Pub. Date: Jun. 22, 2017

(54) ARTIFICIAL LUNG AND ARTIFICIAL HEART-LUNG CIRCUIT DEVICE

(71) Applicant: TERUMO KABUSHIKI KAISHA, Tokyo (JP)

(72) Inventors: Takashi Saito, Elkton, MD (US); Eisuke Sasaki, Elkton, MD (US)

(21) Appl. No.: 15/447,530

(22) Filed: Mar. 2, 2017

Related U.S. Application Data
(63) Continuation of application No. PCT/JP2015/076321, filed on Sep. 16, 2015.

(30) Foreign Application Priority Data
Sep. 30, 2014 (JP) 2014-201780

(51) Int. Cl. A61M 1/16

(52) U.S. Cl. CPC A61M 1/1698; A61M 2205/366

(57) ABSTRACT

An artificial lung has a gas exchanger portion with an external circulation-type gas exchanging hollow fiber membrane bundle and a heat exchanger portion with a heat exchanging element. Various design parameters of the gas exchanger and heat exchanger portions are configured to obtain a predetermined performance factor. In one embodiment, the design parameters are adjusted so that when a heat exchange performance coefficient of the artificial lung, a blood filling amount of the artificial lung, and a blood side pressure loss of the artificial lung are measured, they result in a performance factor equal to or greater than 1.5, wherein the performance factor is defined as heat exchange performance coefficient/(blood filling amount×blood side pressure loss).

### CROSS REFERENCE TO RELATED APPLICATIONS

This application is a continuation of PCT Application No. PCT/JP2015/076321, filed Sep. 16, 2015, based on and claiming priority to Japanese application no. 2014-201780, filed Sep. 30, 2014, both of which are incorporated herein by reference in their entirety.

### TECHNICAL FIELD

The present invention relates to design and performance evaluation of an artificial lung.

### BACKGROUND OF THE INVENTION

An artificial lung is an artificial organ which supplies oxygen to blood and removes carbon dioxide gas from blood, to replace or supplement the functions of a biological lung. Currently, an external circulation-type of artificial lung or oxygenator using a hollow fiber membrane is mainly adopted. Typically, the artificial lung can be used for up to several hours to support heart surgery during which the heart is stopped, wherein a so-called cardiotomy is used for forming an extracorporeal blood circulation circuit while the biological lung is typically not perfused. In addition, the artificial lung can also be utilized as a respiration assisting apparatus or a percutaneous cardiopulmonary assisting apparatus for a patient of acute pulmonary insufficiency or cardiac insufficiency.

In the artificial lung used in extracorporeal circulation of blood, since blood is guided out of the body of a patient, heat is taken from the blood. Therefore, when blood is returned to the patient, the blood is required to be rewarmed to be near the body temperature. In addition, a conventional extracorporeal circulation method sometimes employs a technique wherein the blood temperature is lowered so as to restrain metabolism of cells while the brain and central nerves are protected. Therefore, the artificial lung is normally provided with a heat exchanging element for adjusting a blood temperature.

The extracorporeal circulation circuit such as an artificial heart-lung circuit device is prepared for use by being filled with a priming solution or the like. A lactate Ringer solution or the like is used as the priming solution, and blood becomes diluted by a corresponding filling amount (blood filling amount) of the circuit. The diluted blood leads to a reduced effectiveness and a corresponding burden to a patient. In order to reduce the effects of diluted blood, a blood transfusion can be performed. However, the blood transfusion carries a risk of a physical burden or an infection occurring in a patient. Therefore, a reduction of the blood filling amount of the extracorporeal circulation circuit is desired. As a result thereof, in the artificial lung which is one of the components of the extracorporeal circulation circuits, reduction of the blood filling amount is desirable for the artificial lung as well.

In the design and performance evaluation of an artificial lung, it is favorable to have gas exchange performance of the artificial lung and heat exchange performance of the artificial lung to be as high as possible, and it is favorable to have the blood filling amount of the artificial lung and a blood side pressure loss of the artificial lung to be as low as possible. For example, in a case where the blood filling amount is reduced so as to minimize a burden to a human body, improvements such as reducing the surface area of a gas exchanging hollow fiber membrane or the heat exchanging element and acquiring high performance through a small surface area by densely installing the gas exchanging hollow fiber membrane or the heat exchanging element may be required. However, when the surface area of the gas exchanging hollow fiber membrane or the heat exchanging element is reduced, the gas exchange performance and the heat exchange performance thereof are deteriorated. When the gas exchanging hollow fiber membrane or the heat exchanging element are densely packed, there is concern of an increase of a blood side pressure loss and an increase of damage to the blood cells.

In the related prior art, each of the elements of the artificial lung has been improved independently from other elements (see, e.g., Japanese publication JP-T-2014-514963), but there is a demand for a technology in which such various elements are united and the elements can be evaluated and examined regardless of the type and the size of the artificial lung.

### SUMMARY OF THE INVENTION

### Technical Problem

There is provided a technology of designing and evaluating an artificial lung in which performance of each of elements of the artificial lung can be uniformly examined and a generally excellent performance can be exhibited, and there is provided an artificial lung exhibiting the generally excellent performance acquired by using this method.

### Solution to Problem

The inventors have found that regardless of the difference of the type and the structure of the details of an artificial lung, when gas exchange performance, a heat exchange performance coefficient, a blood filling amount, and a blood side pressure loss are measured, performance factors of the artificial lung can be acquired through combinations of the measured results, and regardless of the type and the structure thereof, the artificial lung can be generally evaluated and compared through the acquired performance factors. Thus, the present invention has been realized.

According to a first aspect of the present invention, and artificial lung includes a gas exchanger portion with an external circulation-type gas exchanging hollow fiber membrane bundle and a heat exchanger portion with a heat exchanging element. The artificial lung is configured such that a performance factor is equal to or greater than 0.15, wherein the performance factor is determined according to a formula:

heat exchange performance coefficient of the artificial lung/(blood filling amount of the artificial lung×blood side pressure loss of the artificial lung).

As used herein, heat exchange performance coefficient of the artificial lung is comprised of a dimensionless (i.e., scalar or absolute) number in accordance with ISO 7199 5.4.2, blood filling amount of artificial lung is measured in the unit [mL] (1 mL=1 L/1,000) conforming to ISO 7199 5.3.3, and blood side pressure loss of artificial lung is measured in the unit [mmHg/(L/min)].

According to a second aspect of the invention, an artificial lung includes a gas exchanger portion with an external circulation-type gas exchanging hollow fiber membrane bundle, and a heat exchanger portion with a heat exchanging element. The artificial lung is configured such that a performance factor ranges from 1.5 to 2.5, wherein the performance factor is determined according to a formula:

(gas exchange performance×heat exchange performance coefficient)/(blood filling amount×blood side pressure loss).

As used herein, the heat exchange performance coefficient of the artificial lung, the blood filling amount of the artificial lung, and the blood side pressure loss of the artificial lung are the same as those described above. Gas exchange performance of artificial lung can be derived according to a method disclosed in Artificial Organ 16(1), 654-657 (1987), “Examination of Artificial Lung Performance Evaluation Method” by Nogawa, wherein the gas exchange performance is preferably determined in the unit [L/min].

In another embodiment, a performance factor is defined according to an Expression heat exchange performance coefficient of heat exchanger portion/(blood filling amount of heat exchanger portion×blood side pressure loss of heat exchanger portion). The artificial lung is configured to provide a value of this performance factor equal to or greater than 1.5.

The gas exchange performance is configured for a blood flow rate at which blood having oxygen saturation of 97.5% can be acquired when the artificial lung is operated with hemoglobin concentration of 12.0 g/dL and oxygen saturation of 50% in venous blood.

### Advantageous Effect of Invention

In the artificial lung according to the present invention, regardless of the type and the difference of the structure of the details of the artificial lung, evaluation can be generally performed by using performance factors as defined above, and thus, a generally excellent artificial lung performance can be acquired.

### BRIEF DESCRIPTION OF DRAWINGS

FIGS. 1A and 1B are cross-sectional views illustrating examples of structure of artificial lungs.

FIG. 2 is a schematic view describing an artificial heart-lung circuit device.

### DETAILED DESCRIPTION OF PREFERRED EMBODIMENTS

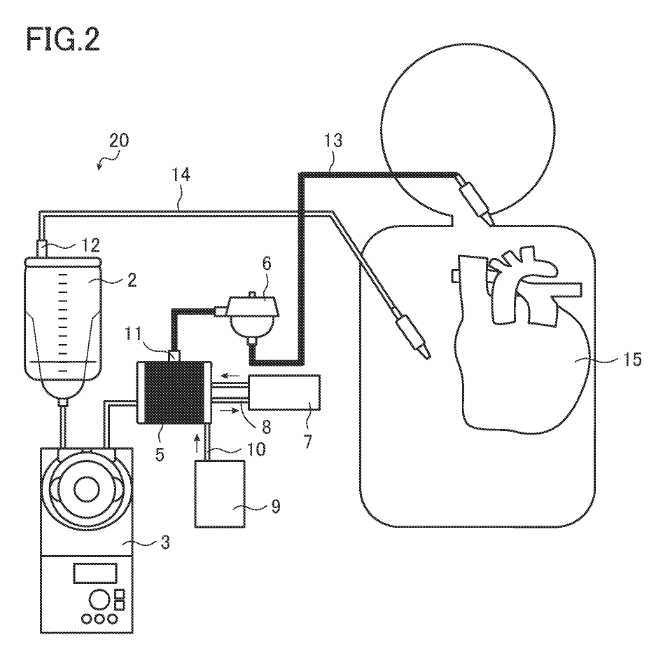

First, description will be given regarding an artificial heart-lung circuit device 20 of the present invention illustrated in FIG. 2. FIG. 2 is a conceptual circuit diagram of an example of an artificial heart-lung circuit device of the present invention and illustrates an artificial lung in which a heat exchanging element is internally mounted. A heat exchanger portion (not illustrated) that is provided with the heat exchanging element and a gas exchanger portion (not illustrated) that is provided with a hollow fiber membrane bundle are included within an artificial lung 5. The artificial heart-lung perfusion circuit 20 includes a venous blood reservoir 2 provided with a blood retention portion which retains blood and from which blood inside thereof can flow out by a blood supply pump 3. The artificial lung 5 is connected to a blood outlet port of the blood supply pump 3, and an artery filter 6 is provided on a downstream side of the artificial lung 5. A heat medium supplier 7 is provided which supplies a heat medium to the heat exchanger portion of the artificial lung 5, and a heat medium 8 supplied from the heat medium supplier 7 circulates in the artificial lung 5, thereby performing heat-exchanging. In addition, oxygen-containing gas 10 is supplied from an oxygen-containing gas supplier 9 to the inside of a hollow fiber membrane of the artificial lung 5.

Specifically, the artificial heart-lung perfusion circuit 20 interconnects the venous reservoir 2, a blood removal (venous blood) line 14 which is connected to a blood inlet port 12 of the vein reservoir 2 and through which blood removed from a heart 15 is received, and a blood supply (arterial blood) line 13 through which blood is supplied to the heart 15 through a blood outlet port 11 of the artificial lung 5 via the artery filter 6.

According to the present invention, in evaluation of an artificial lung, it is favorable to consider various factors, such as, gas exchange performance of the artificial lung [measured in L/min] and a heat exchange performance coefficient of artificial lung [measured as an absolute (i.e., dimensionless or scalar) number], which are preferably made as large as possible. Furthermore, it is desirable to have a blood filling amount of artificial lung [measure in mL] (1 mL=1 L/1,000) and a blood side pressure loss of artificial lung [measured in mmHg/(L/min)] to be as small as possible. In order to optimize performance of an artificial lung, the present invention defines a performance factor Fp of the gas exchanger portion and the heat exchanger portion of the artificial lung through the following Expression:

Fp=(gas exchange performance [L/min]×heat exchange performance coefficient [absolute number])/(blood filling amount [L]×blood side pressure loss [mmHg/(L/min)]).

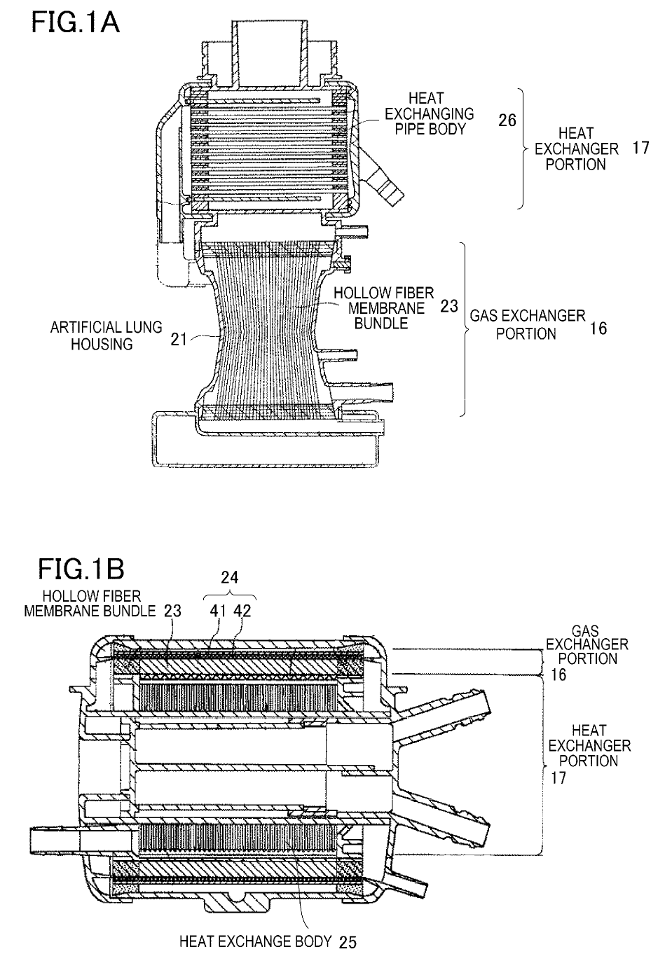

FIG. 1 illustrates cross-sectional views of two examples of external circulation-type artificial lungs in which hollow fiber membranes are used. In the artificial lung illustrated in FIG. 1A, a tubular body-shaped artificial lung housing 21 is formed and a hollow fiber membrane bundle 23 is internally accommodated, thereby configuring a gas exchanger portion 16. Above the gas exchanger portion 16, there is provided a heat exchanger portion 17 in which multiple heat exchanging pipe bodies 26 serving as the heat exchanging elements are disposed and blood flows in the heat exchanging pipe bodies 26 and is subjected to heat exchange.

In the artificial lung illustrated in FIG. 1B, a housing in its entirety has a cylindrical shape. A filter member 41 and an exhaust hollow fiber membrane layer 42 configure air bubble removal means 24 which functions as an artery filter are internally provided in a concentric cylindrical manner from a side close to the side wall of the housing, and the hollow fiber membrane bundle 23 is internally disposed in a concentric circle manner, thereby configuring the gas exchanger portion 16. A heat exchange body 25 serving a heat exchanging element is disposed further inside, thereby configuring the heat exchanger portion 17.

As described in the examples of FIG. 1, the artificial lung of the present invention is provided with at least the gas exchanger portion 16 and the heat exchanger portion 17. Examples of the gas exchanger portion include a region which is configured to have the gas exchanging hollow fiber membrane bundle 23 disposed in the artificial lung 5, and an external circulation blood portion surrounding the hollow fiber membrane bundle 23 and allowing blood to flow therein, and in which the hollow fiber membrane bundle 23 and the blood come into contact with each other. The heat exchanger portion 17 is configured to have the heat exchanging element in which blood flows, and a heat medium circulation chamber surrounding the heat exchanging element.

Description will be given regarding a method of measuring each of the factors for calculating a performance factor for optimizing performance of the artificial lung as a whole.

Gas exchange performance of the artificial lung can be measured in the units of [L/min] according to the method described in Artificial Organ 16(1), 654-657 (1987), “Examination of Artificial Lung Performance Evaluation Method” by Nogawa. In particular, an artificial lung is considered to be a black box in a Step A of this method. Examples of inputs to the artificial lung include Sv (the rate of oxygenated hemoglobin in venous blood), Hb (the hemoglobin concentration of test blood), and QB (the blood flow rate). As an output from the artificial lung, SAO2 (the rate of oxygenated hemoglobin of the blood supply line) can be examined. When the output with respect to all of the inputs is measured, the gas exchange performance of the artificial lung can be evaluated. However, it has been empirically learned that the conditions are not complete independent variables and are correlated to each other. Therefore, the evaluation can be simplified by reducing the independent variables.

A Step B considers a quantity Hb×QB×(1−SvO2), which can be referred to as the oxygen vacancy quantity (the quantity of oxygen which can be received) with respect to diffusion. When the quantities are equal to each other between different types of artificial lungs, it is assumed that the diffusion states in the artificial lungs are similar to each other (called a diffusion stratum similarity theory).

In a Step C, the above-referenced theory is broadened to a turbulence-type artificial lung, and the performance is improved due to an increase of the flow rate and an increase of the Reynold's number. Therefore, the phenomenon is described based on a varying diffusion coefficient D. Since Hb and Re have a positive correlationship, a correction coefficient of Re is taken in both Hb and QB, thereby acquiring a grounded theoretical expression broadened to the turbulence-type artificial lung.

Step D proceeds according to ISO 7199 5.4.1, wherein (1−SAO2)=97.5% and Hb=12 g/dL, X-axis: (1−SvO2)×Hb×QB, and Y-axis: (1−SvO2) are established. Since (1−SvO2) is determined by the Y-axis, (1−SvO2) is taken from the X-axis. Furthermore, the index of QB is removed. As a result thereof, a curve having the X-axis: (1−SvO2) and the Y-axis: QB can be acquired.

It is extremely difficult to perform an experiment for drawing a graph of Step D. However, in Step E, since experimental data of (1−SAO2)=97.5% can be calculated through an experiment other than (1−SAO2)=97.5% by using a variable transformation method, a graph (not illustrated) can be drawn. The variable transformation method used here is a method in which in a case where a value expressed by a complex function ƒ(x) is unknown, a variable acquired through the experiment other than (1−SAO2)=97.5% transformed to a variable of the experimental data of (1−SAO2)=97.5% such that x (unknown variable) of the function ƒ(x) does not change.

In other words, the gas exchange performance of the artificial lung is defined as a blood flow rate at which oxygen saturation of blood flowing out when blood having the hemoglobin concentration of 12.0 g/dL and the oxygen saturation of 50% flows in the artificial lung becomes 97.5%. As the gas exchange performance of the artificial lung increases, the value of the blood flow rate becomes greater. However, it is difficult to acquire the blood flow rate through an experiment. The reason is that in the calculation of the gas exchange performance, the oxygen saturation on the outlet side (artery side) is an input and the blood flow rate is an output. Meanwhile, in an experiment, the oxygen saturation on the outlet side (artery side) becomes an output and the blood flow rate becomes an input. Thus, by using the variable transformation method disclosed in Artificial Organ 16(1), 654-657 (1987), “Examination of Artificial Lung Performance Evaluation Method” by Nogawa, it is possible to acquire the blood flow rate from several pieces of experimental data, that is, the value of the gas exchange performance. Artificial Organ 16(1), 654-657 (1987), “Examination of Artificial Lung Performance Evaluation Method” by Nogawa can be referred to for more details of the variable transformation method.

A method of measuring gas exchange performance of the present invention will be specifically described. The artificial lung is embedded in an appropriate test circuit, and oxygen gas is supplied to the artificial lung from an oxygen gas supplier. Blood is caused to flow into the artificial lung under conditions of arbitrary oxygen saturation and an arbitrary blood flow rate on an inlet side (vein side), and blood at the entrance and the exit of the artificial lung is taken, thereby acquiring the oxygen saturation. It is desirable that the hemoglobin concentration is 12.0±1 g/dL. It is desirable to set the conditions of the oxygen saturation and the blood flow rate on the inlet side (vein side) such that the oxygen saturation on the outlet side (artery side) does not exceed 99%.

By using the acquired value and the variable transformation method disclosed in Artificial Organ 16(1), 654-657 (1987), “Examination of Artificial Lung Performance Evaluation Method” by Nogawa, a blood flow rate QB is calculated so as to acquire a result having the hemoglobin concentration Hb of 12.0 g/dL, the oxygen saturation SvO2 of 50% in venous blood, and the oxygen saturation SAO2 of 97.5% in arterial blood. The blood flow rate QB thereof corresponds to the gas exchange performance.

Specifically, measurement under several conditions is performed in this experiment, and the measurement results are evaluated according to,

SAO2/SvO2=ƒ{(1−SvO2)×Hb(1+cRe)×QB(1−cRe−cQB)} Expression (1)

Using substitution for cRe and cQB, and wherein SAO2=0.975 (that is, 97.5%), SvO2=0.500 (that is, 50%), and Hb=12.0, QB can be obtained. The QB is the gas exchange performance of the present invention.

Heat exchange performance coefficient of the artificial lung can be measured as an absolute, scalar number using the method of ISO 7199 5.4.2. Thus, a heat exchange performance coefficient R is determined as follows.

R=[(temperature Bout of blood at blood outlet port of artificial lung)−(temperature Bin of blood at blood inlet port of artificial lung)]/[(temperature Win of heat medium at heat medium inlet port of heat exchanger portion of artificial lung)−(temperature Bin of blood at blood inlet port of artificial lung)].

As a test solution for a blood flow path, blood which is taken from a cow and is subjected to heparinization is used. A test is performed under conditions in which the temperature Bin of blood at the blood inlet port of the artificial lung is 30±1° C. and the temperature Win of the heat medium at the heat medium inlet port of the heat exchanger portion of the artificial lung is 40±1° C. The blood flow rate is the maximum blood flow rate of the artificial lung. Water is used as the heat medium, and the measurement is performed under the condition in which the flow rate of the heat medium is 10 L/min. In the calculation of the heat exchange performance coefficient of the artificial lung, the total sum of the performance of heat exchange in the gas exchanger portion and heat exchange in the heat exchanger portion is measured as the heat exchange performance coefficient. Accordingly, the measurement value can be acquired regardless of the type and the configuration of the artificial lung.

Blood filling amount of the artificial lung can be measured in units of [mL] using the method described in ISO 7199 5.3.3, wherein the dry weight of the artificial lung before being filled is measured on a scale. Thereafter, the artificial lung is filled with a physiological salt solution. The weight of the artificial lung after being filled is measured on the scale again, and the filling amount is calculated through the following expression:

Filling amount=(weight after being filled−dry weight)/(proportion 1.006 of physiological salt solution).

Blood side pressure loss of the artificial lung can be measured using units of [mmHg/(L/min)]. The artificial lung to be measured is embedded in an appropriate circuit, and the pressures at the blood inlet port and the blood outlet port are measured, thereby calculating the pressure loss through the following expression wherein the blood flow rate is measured at the maximum blood flow rate of the artificial lung to be measured:

Pressure loss=(inlet port pressure−outlet port pressure)/(maximum blood flow rate).

The blood temperature is set to 37±1° C. and the hematocrit value is set to 35±1%. The test solution is measured by using blood of a cow. The measurement value can be acquired regardless of the type and the configuration of the artificial lung product.

When the performance factor Fp of commercially available artificial lungs currently on the market is calculated by using the performance factors defined above, the result of Table 1 below is acquired.

TABLE 1

| | Product A | Product B | Product C | Product D |
|---|---|---|---|---|
| Fp | 1.0 | 1.0 | 0.5 | 1.2 |

Other artificial lungs as disclosed in Examples 1 to 3 are described in greater detail below. The gas exchange performance of the artificial lung [L/min], the heat exchange performance coefficient of artificial lung [absolute, scalar number], the blood filling amount of artificial lung [mL] (1 mL=1 L/1,000), and the blood side pressure loss of artificial lung [mmHg/(L/min)] are measured through the measurement method disclosed above and Fp is calculated, thereby acquiring the result of Table 2 below. The artificial lungs of Examples 1 to 3 use arterial filtering for internally mounted-type artificial lungs. However, in the method of calculating the performance factor of the present invention, regardless of the form of internally mounting or externally mounting the artery filter, the performance factor can be calculated, evaluated, and compared to each other. Therefore, the method can be applied to various types of artificial lungs. In other words, as described in the measurement method above, in a case where the artery filter is internally mounted, the gas exchange performance of the artificial lung, the heat exchange performance coefficient of the artificial lung, and the blood side pressure loss of the artificial lung are the results reflecting the internally mounted state. Regarding the blood filling amount of the artificial lung, the portion of the artery filter can be relatively ignored with respect to the total blood filling amount. Therefore, the measurement can be performed whether or not the artery filter is internally mounted.

TABLE 2

| Item | Unit | Example 1 | Example 2 | Example 3 |
|---|---|---|---|---|
| Gas exchange performance | L/min | 8.2 | 8.2 | 8.2 |
| Heat exchange performance | — | 0.58 | 0.57 | 0.59 |
| coefficient | | | | |
| Blood side pressure loss | mmHg/ | 15.7 | 15.7 | 15.7 |
| | (L/min) | | | |
| Blood filling amount | mL | 150 | 160 | 180 |
| Performance factor Fp | — | 2.0 | 1.9 | 1.7 |

According to the result of Table 2, it is verified that the artificial lung having the performance factor of Fp of the present invention ranging from 1.5 to 2.5 and preferably ranging from 1.7 to 2.1 has a sufficiently high performance factor compared to the performance factor of conventional artificial lungs available on the market, and when the performance factor of Fp is used during design and evaluation of an artificial lung, a generally excellent artificial lung can be designed, and the performance of the artificial lung can be appropriately evaluated.

According to a second embodiment of the invention, a Simplified Performance Factor of the Artificial Lung in Its Entirety, referred to herein as Fp3 can be used as an alternative performance factor for design and evaluation of an artificial lung device. In this embodiment, simplified measurement and calculation is based on defining a performance factor Fp3 having only the heat exchange performance coefficient of artificial lung [absolute number] as the numerator and blood filling amount of artificial lung [L]×blood side pressure loss of artificial lung [mmHg/(L/min)] as the denominator. The Expression of Fp3 is as follows:

Fp3=(heat exchange performance coefficient of artificial lung [absolute number])/(blood filling amount of artificial lung [L]×blood side pressure loss of artificial lung [mmHg/(L/min)]).

According to the result of Table 3, it can be understood that an artificial lung having Fp3 equal to or greater than 0.15 and preferably equal to or greater than 0.20 has a sufficiently high performance factor compared to the performance factor of the artificial lungs available on the market, and when the performance factor of Fp3 is used in design and evaluation of the artificial lung, a generally excellent artificial lung can be designed, and the performance of the artificial lung can be appropriately evaluated through the simplified measurement and calculation.

TABLE 3

| Item | Unit | Example 1 | Example 2 | Example 3 |
|---|---|---|---|---|
| Heat exchange performance | — | 0.58 | 0.57 | 0.59 |
| coefficient | | | | |
| Blood side pressure loss | mmHg/ | 15.7 | 15.7 | 15.7 |
| | (L/min) | | | |
| Blood filling amount | mL | 150 | 160 | 180 |
| Performance factor Fp3 | — | 0.25 | 0.23 | 0.21 |

According to a third embodiment of the invention, an Examination with Only Heat Exchanger Portion using a performance factor referred to herein as Fp4 is used. Regarding the artificial lungs of Examples 1 to 3 of the present invention, when an examination is performed while having only the heat exchanger portion as the subject, a performance factor Fp4 is examined while having heat exchange performance coefficient of the heat exchanger portion [absolute number] as the numerator and blood filling amount of heat exchanger portion [L]×blood side pressure loss of heat exchanger portion [mmHg/(L/min)] as the denominator:

Fp4=(heat exchange performance coefficient of heat exchanger portion [absolute number])/(blood filling amount of heat exchanger portion [L]×blood side pressure loss of heat exchanger portion [mmHg/(L/min)]).

The blood filling amount of the heat exchanger portion indicated herein is calculated based on the volume of only a blood circulation chamber of the heat exchanger portion disposed in the artificial lung 5, and the blood side pressure loss of the heat exchanger portion is calculated by dividing the pressure difference between blood flowing into the heat exchanger portion and blood flowing out from the heat exchanger portion, by the maximum blood flow rate. According to Fp4, only the heat exchanger portion is taken as the subject, a heat exchanger portion excellent in the heat exchange performance can be designed, and the performance of the heat exchanger portion can be appropriately evaluated. Therefore, as a result, it is possible to design and evaluate an excellent artificial lung.

Description will be given regarding a method of measuring each of the factors for calculating the performance factor while focusing on only the heat exchanger portion of the artificial lung. For the heat exchange performance coefficient of heat exchanger portion, the same measurement method and the same measurement values as those of the above-described heat exchange performance coefficient of the artificial lung in its entirety are used. For the blood filling amount of artificial lung with only heat exchanger portion, since the unit [mL] and each of the dimensions of a heat exchange element of the heat exchanger portion are known, in a case where blood flows in the heat exchange element, the volume thereof becomes the blood filling amount. In a case where the heat medium flows inside the heat exchange element and the surroundings thereof is filled with blood, the blood filling amount can be calculated through a method of estimating the filling amount based on a portion from which the volume of the heat exchange element is subtracted.

Blood side pressure loss of heat exchanger portion is determined based on the unit [mmHg/(L/min)]. The pressures at the blood inlet port and the blood outlet port of the heat exchanger portion are measured, and the pressure loss is calculated through the following expression, wherein the blood flow rate is measured at the maximum blood flow rate of the artificial lung product to be measured:

Pressure loss=(inlet port pressure−outlet port pressure)/(maximum blood flow rate).

The blood temperature is set to 37±1° C. and the hematocrit value is set to 35±1%. The test solution is measured by using blood of a cow.

TABLE 4

| Item | Unit | Example 1 | Example 2 | Example 3 |
|---|---|---|---|---|
| Heat exchange performance | — | 0.58 | 0.57 | 0.59 |
| coefficient | | | | |
| Blood side pressure loss | mmHg/ | 7.9 | 6.2 | 6.7 |
| | (L/min) | | | |
| Blood filling amount | mL | 35 | 60 | 70 |
| Performance factor Fp4 | — | 2.1 | 1.5 | 1.3 |

Meanwhile, when the performance factor Fp4 of the artificial lung with only the heat exchanger portion currently on the market is calculated by using the similar performance factor Fp4, the result of Table 5 below is acquired.

TABLE 5

| | Product A | Product B | Product C |
|---|---|---|---|
| Fp4 | 1.3 | 1.4 | 1.0 |

According to the results of Tables 4 and 5, when Fp4 exceeds 1.4, is preferably equal to or greater than 1.5, and is more preferably equal to or greater than 2.0, compared to the conventional products in the related art, it is possible to have a sufficiently high performance factor. When being combined with the gas exchanger portion having the gas exchange performance of the present invention, an artificial lung having an excellent performance factor can be obtained. When the performance factor Fp4 of the present invention is used, the performance of the artificial lung with only the heat exchanger portion can be appropriately designed and evaluated as the subject. Therefore, as a result, it is possible to design and evaluate an artificial lung provided with the heat exchanger portion having excellent heat exchange performance.

### EXAMPLES

The present invention will be specifically described below by using examples configured in a manner that satisfies the performance factors in the manner indicated. However, the present invention is not limited to the artificial lung and the manufacturing method of these examples.

In Examples 1 to 3, in order to design an artificial lung having a performance factor Fp more excellent than the performance factor Fp of the artificial lung currently on the market, shown in Table 1, the below-described improvement of the gas exchanger portion and the heat exchanger portion is designed and specified.

A gas exchanging hollow fiber is reduced in diameter, and the disposition of the hollow fiber membrane bundle is examined. In addition, a heat exchanging resin tube is employed as the heat exchanging element, and the material thereof and the heat-transfer area thereof are adjusted, thereby examining the disposition of the heat exchanging resin tube. The details are disclosed in Table 6.

The blood filling amount of the artificial lung in its entirety is designed so to be preferably equal to or smaller than 200 mL and to be more preferably equal to or smaller than 190 mL. The blood filling amount with only the gas exchanger portion is designed so to be preferably equal to or smaller than 65 mL and to be more preferably equal to or smaller than 60 mL.

All of Examples 1 to 3 adopt artery filter internally mounted-type artificial lungs. In this case, the blood filling amount of a portion of the artery filter can be ignored with respect to the total blood filling amount. Therefore, the performance factors calculated in Examples 1 to 3 are not influenced by the artery filter.

As a result of the examinations, each of the elements with respect to blood becomes highly dense and an artificial lung having a low blood filling amount and high performance can be designed and evaluated.

TABLE 6

| Item | Example 1 | Example 2 | Example 3 |
|---|---|---|---|
| Hollow fiber bundle | Small | Small | Small |
| | diameter | diameter | diameter |
| | polypropylene | polypropylene | polypropylene |
| | hollow fiber | hollow fiber | hollow fiber |
| | bundle | bundle | bundle |
| Gas exchange area | 1.9 m2 | 1.9 m2 | 1.9 m2 |
| Gas exchanger portion | Hollow fiber | Hollow fiber | Hollow fiber |
| | bundle | bundle | bundle |
| | winding | winding | winding |
| Heat exchange element | Resin tube | Resin tube | Resin tube |
| Heat-transfer area | 0.49 m2 | 0.44 m2 | 0.6 m2 |
| Heat exchanger portion | winding | Bamboo | Bamboo |
| | | blind | blind |
| | | winding | winding |
| Presence of artery filter | Present | Present | Present |
| internally mounted | | | |

An artificial lung of the present invention, regardless of the difference of the type and the structure of the details thereof, gas exchange performance, a heat exchange performance coefficient, a blood filling amount, and a blood side pressure loss are measured and performance factors of the artificial lung can be acquired through combinations of the measured results. The artificial lung can be generally evaluated and compared through the acquired performance factors. Therefore, it is possible to generally evaluate and compare the artificial lungs suitable for various types of methods and structure such as a pediatric artificial lung, an extracorporeal circulation circuit used during cardiotomy, a respiration assisting apparatus for a patient of acute pulmonary insufficiency or cardiac insufficiency, and a percutaneous cardiopulmonary assisting apparatus. Thus, it is possible to expect that the present invention is effectively used in designing and manufacturing an artificial lung which can exhibit excellent performance.

What is claimed is:

1. An artificial lung comprising:
a gas exchanger portion with an external circulation-type gas exchanging hollow fiber membrane bundle; and
a heat exchanger portion with a heat exchanging element;
wherein the hollow fiber membrane bundle and the heat exchanging element are configured to provide an artificial lung performance factor equal to or greater than 0.15; and
wherein the performance factor is determined by a heat exchange performance coefficient of the artificial lung, a blood filling amount of the artificial lung, and a blood side pressure loss of the artificial lung according to a formula:
heat exchange performance coefficient/(blood filling amount×blood side pressure loss.

2. An artificial lung comprising:
a gas exchanger portion with an external circulation-type gas exchanging hollow fiber membrane bundle; and
a heat exchanger portion with a heat exchanging element;
wherein the hollow fiber membrane bundle and the heat exchanging element are configured to provide an artificial lung performance factor between 1.5 and 2.5; and
wherein the performance factor is determined by a gas exchange performance of the artificial lung, a heat exchange performance coefficient of the artificial lung, a blood filling amount of the artificial lung, and a blood side pressure loss of the artificial lung according to a formula:
(gas exchange performance×heat exchange performance coefficient)/(blood filling amount×blood side pressure loss.

3. The artificial lung of claim 2 wherein the gas exchange performance is configured according to a blood flow rate at which blood having an oxygen saturation of 97.5% is obtained when the artificial lung is operated with a hemoglobin concentration of 12.0 g/dL and an oxygen saturation of 50% in venous blood.

4. An artificial lung comprising:
a gas exchanger portion with an external circulation-type gas exchanging hollow fiber membrane bundle; and
a heat exchanger portion with a heat exchanging element;
wherein the hollow fiber membrane bundle and the heat exchanging element are configured to provide an artificial lung performance factor equal to or greater than 1.5; and
wherein the performance factor is determined by a heat exchange performance coefficient of the heat exchanger portion, a blood filling amount of the heat exchanger portion, and a blood side pressure loss of the heat exchanger according to a formula:
heat exchange performance coefficient of heat exchanger portion/(blood filling amount of heat exchanger portion×blood side pressure loss of heat exchanger portion).
